(19) United States
(12) Patent Application Publication
(10) Pub. No.: US 2004/0259128 A1
(43) Pub. Date: Dec. 23, 2004

(54) Methods and compositions for detecting the presence of target nucleic acids in a sample

(76) Inventors: Glenn Kawasaki, Seattle, WA (US); Bruce M. Travis, Seattle, WA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/799,925

(22) Filed: Mar. 11, 2004

Related U.S. Application Data
(60) Provisional application No. 60/532,699, filed on Dec. 24, 2003.
(60) Provisional application No. 60/457,527, filed on Mar. 24, 2003.

(51) Int. Cl. C12Q 1/68; C07H 21/02

(52) U.S. Cl. 435/6; 536/23.1

(57) ABSTRACT

Methods and compositions for detecting the presence, e.g., quantitatively, of a target nucleic acid, such as an siRNA, in a sample are provided. In the subject methods, a sample is contacted with at least two different ligation domains, which may be present on separate nucleic acids (e.g., oligonucleotides) or on the same complex, e.g., Combined Oligo, to produce a reaction mixture, where each of the different ligation domains includes a domain complementary to a different region of the target nucleic acid. The ligation domains of any resultant ligation domain/target nucleic acid complexes are then ligated to produce a pseudotarget nucleic acid. The presence of any resultant pseudotarget nucleic acids in the reaction mixture is then determined in order to detect the target nucleic acid in the sample. Also provided are systems and kits that find use in practicing the subject methods. The subject invention finds use in a variety of applications, including therapeutic applications.

### CROSS-REFERENCE TO RELATED APPLICATIONS

Pursuant to 35 U.S.C. § 119 (e), this application claims priority to the filing date of the U.S. Provisional Patent Application Ser. No. 60/532,699 filed Dec. 24, 2003 and to the filing date of United States Provisional Patent Application Serial No. 60/______filed on Mar. 24, 2003; the disclosures of which are herein incorporated by reference.

### INTRODUCTION

1. Field of the Invention

The field of this invention is nucleic acid detection.

2. Background of the Invention

A multitude of biotechnology applications exists that require the detection of one or more specific nucleic acids in a complex mixture of nucleic acids. Such applications include both research and clinical, e.g., diagnostic and therapeutic, applications. Representative specific types of nucleic acid detection protocols that have been developed include: blotting protocols, such as Northern and Southern blot protocols; array based nucleic acid detection protocols; and PCR based detection protocols.

Despite the large number of nucleic acid detection protocols that have been developed, there continues to be interest in the development of new nucleic acid detection protocols. Of particular interest would be the development of a highly sensitive ribonucleic acid detection protocol that could quantitatively detect the presence of short RNA target molecules in complex mixtures. The present invention satisfies this need.

Relevant Literature

Published U.S. Patent Applications of interest include: 20030119004; 20030190646; 20030082584; 20030032024 and 20020177133. Articles of interest include: Hsuih et al., J. Clinical Microbiology (March, 1996) 34: 501-507 and Nilsson et al., Nuc. Acids Res. (2001) 29: 578-581.

### SUMMARY OF THE INVENTION

Methods and compositions for detecting the presence, e.g., quantitatively, of a target nucleic acid, such as a siRNA, in a sample are provided. In the subject methods, a sample is contacted with at least two different ligation domains, which domains may be present on the same nucleic acid or different nucleic acids, to produce a reaction mixture, where each of the different ligation domains is complementary to a different region, e.g., adjacent regions of the target nucleic acid. The ligation domains of any resultant ligation domain/target nucleic acid complexes are then ligated to each other to produce a pseudotarget nucleic acid. The presence of any resultant pseudotarget nucleic acids in the reaction mixture is then determined in order to detect the target nucleic acid in the sample. Also provided are systems and kits that find use in practicing the subject methods. The subject invention finds use in a variety of applications, including therapeutic applications.

### BRIEF DESCRIPTION OF THE FIGURES

FIG. 1 shows a scheme of the inventive process steps. Specifically, FIG. 1 shows a 21 mer target sequence (siRNA antisense strand) being annealed to two ligation domains present on two different oligonucleotides (TLig5c and TLig3c) corresponding to the 5′ and 3′ ends of the target siRNA sequence, respectively. Both oligos have sequences that extend past the 3′ and 5′ ends representable of the siRNA antisense strand. The ligation step joins the two oligonucleotides. The PCR step uses primers appropriate for regions of the two oligos that extend beyond the 3′ and 5′ termini on the target surrogate. The net effect is to create a target surrogate, through a ligation process, that is of sufficient length to be analyzed and quantified by appropriate means whereas the target siRNA is generally too short for quantitative analysis. In the absence of the annealing and ligation steps, PCR amplification is reduced to very low background levels.

FIG. 2 breaks down the process and shows the target siRNA1 antisense sequence bolded on the top strand and where the two oligonucleotides (modified (5′-Phos)Tlig3c and Tlig5c) line up to the antisense region. There are two shorter PCR primers (labeled TL3c and TL5c) that anneal to their respective oligonucleotide sequences, but generally at regions outside of the regions of the siRNA antisense. While it is not required that the two PCR primers line up to sequences that completely correspond to regions outside of the siRNA antisense sequence, some extension is needed in order to make the target surrogate of sufficient length so as to extend the length of the siRNA antisense target.

FIG. 3 shows the result of the ligation step (before PCR amplification) wherein the siRNA antisense strand (called Primary Target) on top is lengthen by the “secondary target surrogate on the bottom.” This illustrates that the purpose of the surrogate target is to have a complementary sequence to the target sequence and lengthen the target sequence in both the 5′ and 3′ directions. Moreover, the regions where the target sequence is lengthened on the secondary target surrogate can have regions designed for both PCR primer amplification and for attaching a detection probe in order to best quantify the surrogate targets presence.

FIG. 4 shows the secondary target surrogate on top with two primers aligned to the secondary surrogate's outer 3′ and 5′ ends.

FIG. 5 shows the primary target (siRNA) on top, and the secondary surrogate target on the second line composed of the two oligonucleotide sequences that are ligated together. Below the secondary surrogate target are the two primers for amplifying the secondary surrogate target and two additional sequences aligned to more of a middle region of the secondary surrogate target. The two middle sequences are called exciter probe and emitter probe; and they provides a means (e.g., via fluorescent resonance energy transfer (FRET)) for determining the presence and quantity of the secondary surrogate target by aligning to sequence regions on the secondary surrogate target that are outside of those regions on the secondary surrogate target that correspond to the sequence regions of the primary target siRNA antisense.

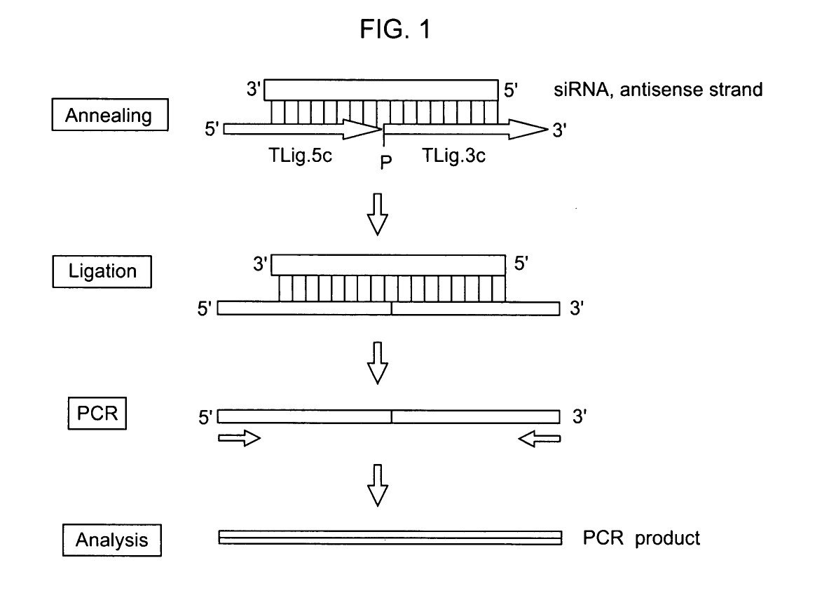

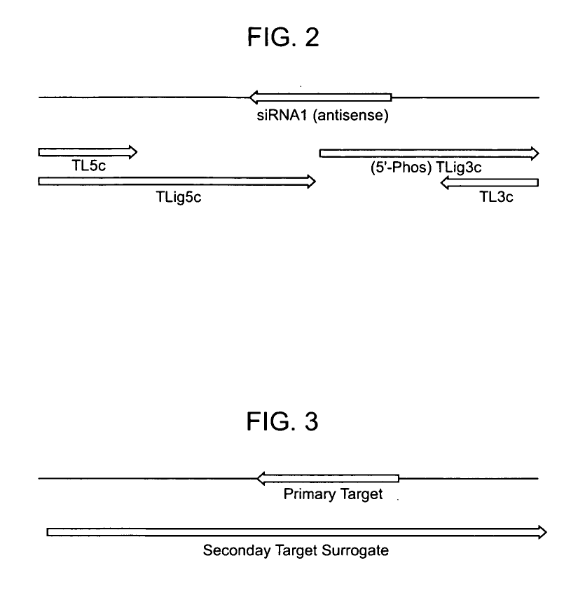

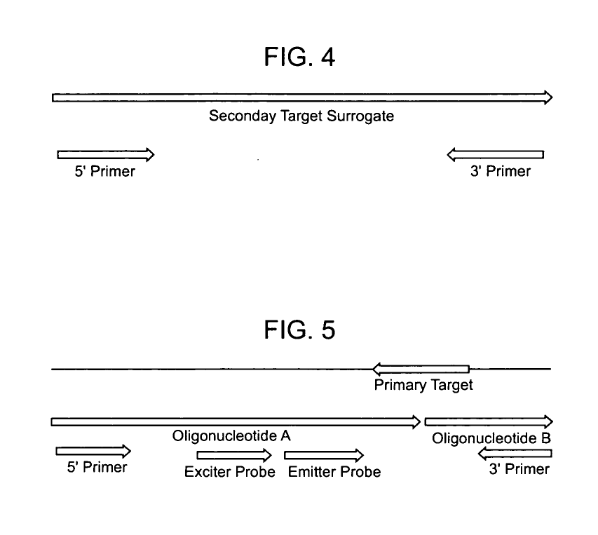

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. As such, the term nucleic acid as used herein includes nucleic acids made up solely of nucleotides found in naturally occurring nucleic acids, as well as peptide nucleic acids, locked nucleic acids, methylated nucleic acids, nucleic acid conjugates, thio-nucleic acids, morpholino nucleic acids, as well as combinations or mixtures thereof.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “stringent assay conditions” refers to conditions under which a probe will hybridize preferentially to its target subsequence, and to a lesser extent to, or not at all to, other sequences. Put another way, the term “stringent assay” or “stringent hybridization conditions” as used herein refers to conditions that are compatible to produce duplexes between complementary binding members, e.g., between probes and complementary targets in a sample, e.g., duplexes of nucleic acid probes, such as target and ligation oligonucleotides.

“Stringent assay” and “stringent hybridization conditions” in the context of nucleic acid hybridization are sequence dependent, and are different under different environmental parameters. See Sambrook, Ausubel, or Tijssen (cited below) for detailed descriptions of equivalent hybridization and wash conditions and for reagents and buffers, e.g., SSC buffers and equivalent reagents and conditions.

The term “stringent conditions” also applies to PCR conditions that reduce background annealing of the PCR primers to unintended DNA or RNA molecules, including the PCR primers themselves, which may lead to primer dimers. Conditions that reduce these mispriming events are well-known by those skilled in the art of gene amplification.

A specific example of stringent assay conditions is provided in the Experimental Section, below.

Stringent assay conditions are conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

### DESCRIPTION OF THE SPECIFIC EMBODIMENTS

Methods and compositions for detecting the presence, e.g., quantitatively, of a target nucleic acid, such as a siRNA, in a sample are provided. In the subject methods, a sample is contacted with at least two different ligation domains, which domains may be present on the same nucleic acid or on different nucleic acids, e.g., oligonucleotides, to produce a reaction mixture. Each of the different ligation domains includes a domain complementary to a different region, e.g., adjacent regions, of the target nucleic acid. The ligation domains of any resultant ligation domain/target nucleic acid complexes are then ligated to each other to produce a pseudotarget nucleic acid. The presence of any resultant pseudotarget nucleic acids in the reaction mixture is then determined in order to detect the target nucleic acid in the sample. Also provided are systems and kits that find use in practicing the subject methods. The subject invention finds use in a variety of applications, including therapeutic applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing the components that are described in the publications which might be used in connection with the presently described invention.

In further describing the subject invention, the subject methods are described first in greater detail, followed by a review of representative applications therefore, as well as a review of representative systems and kits according to the present invention.

Methods

As summarized above, the present invention provides methods of detecting a target nucleic acid in a sample. The target nucleic acid may, in principle be any type of nucleic acid, where the term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) or mixtures of various nucleotides and analogs that can hybridize with naturally occurring nucleic acids in a sequence specific manner analogous to that of two naturally occurring nucleic acids, e.g., can participate in Watson-Crick base pairing interactions. As such, the target nucleic acid may be either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and functionality to the nucleic acid. Such modifications include, but are not limited to, 2′-position sugar modifications, e.g., as found in locked nucleic acids (LNA), 2′-O-methyl RNAs, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine, and the like. Modifications can also include 3′ and 5′ modifications such as capping.

In the broadest sense, the target nucleic acid that is detected by the subject methods may be of any length, where representative length ranges include from about 5 to about 1000 nt or longer, e.g., from about 10 to about 500 nt, from about 15 to about 200 nt, from about 20 to about 100 nt. In certain embodiments, the target nucleic acid is one that is short, e.g., one that ranges from about 5 to about 50 nt, such as from about 10 to about 40 nt, e.g., from about 15 to about 35 nt, where in certain embodiments, the target nucleic acid has a length that does not exceed about 30 nt or even about 25 nt.

In one particular embodiment of interest, the target nucleic acid is a ribonucleic acid (RNA) that does not exceed about 30 nt, and in many embodiments does not exceed about 25 nt, where the target RNA may, in many embodiments, range in length from about 10 to about 30 nt, such as from about 15 to about 25 nt, including from about 20 to about 23 nt.

The target nucleic acid may be single-stranded or double-double stranded, such that in certain embodiments the target nucleic acid is one that is not hybridized to another nucleic acid, while in other embodiments the target nucleic acid is made up of two distinct nucleic acids hybridized to each other.

In certain embodiments the target nucleic acid is a ribonucleic acid, where in certain of these embodiments the target nucleic acid is a siRNA molecule. Particular siRNA molecules of interest are small ribonucleic acid molecules (also referred to herein as interfering ribonucleic acids), i.e., oligoribonucleotides, that are present in duplex structures, e.g., two distinct oligoribonucleotides hybridized to each other, or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt, where the length in certain embodiments is even shorter, see e.g., the representative lengths provided above. Where the target siRNA is a duplex structure of two distinct ribonucleic acids hybridized to each other, the length of the duplex structure typically ranges from about 15 to 30 bp, usually from about 15 to 29 bp, where lengths between about 20 and 29 bps, e.g., 21 bp, 22 bp, 23 bp are of particular interest in certain embodiments. Where the target siRNA is a duplex structure of a single ribonucleic acid that is present in a hairpin formation, i.e., a shRNA, the length of the hybridized portion of the hairpin is typically the same as that provided above for the siRNA molecules.

As indicated above, the invention provides methods of detecting a target nucleic acid in a sample. In the broadest sense, the method may be qualitative or quantitative. As such, where detection is qualitative, the methods provide a reading or evaluation, e.g., assessment, of whether or not the target nucleic acid is present in the sample being assayed. In yet other embodiments, the methods provide a quantitative detection of whether the target nucleic acid is present in the sample being assayed, i.e., an evaluation or assessment of the actual amount of the target nucleic acid in the sample being assayed. In such embodiments, the quantitative detection may be absolute or, if the method is a method of detecting two or more different target nucleic acids in a sample, relative. As such, the term “quantifying” when used in the context of quantifying a target nucleic acid(s) in a sample can refer to absolute or to relative quantification. Absolute quantification may be accomplished by inclusion of known concentration(s) of one or more control nucleic acids and referencing the detected level of the target nucleic acid with the known control nucleic acids (e.g., through generation of a standard curve). Alternatively, relative quantification can be accomplished by comparison of detected levels or amounts between two or more different target nucleic acids to provide a relative quantification of each of the two or more different nucleic acids, e.g., relative to each other.

The subject methods can be employed to detect the presence of one or more target nucleic acids in a variety of different types of samples, including complex samples having large amounts of non-target nucleic acid, where the subject methods provide for detection of the target nucleic acid(s) with high sensitivity. As such, the subject methods are highly sensitive methods of detecting one or more target nucleic acids in a simple or complex sample. The sample that is assayed in the subject methods is typically a sample of nucleic acids, usually RNAs or nucleic acid derivatives thereof, like cDNA, amplified DNA, cRNA, etc., from a physiological source. The physiological source of nucleic acids, e.g. RNAs, will typically be eukaryotic or prokaryotic, with physiological sources of interest including sources derived from single celled organisms such as bacteria and yeast and multicellular organisms, including plants and animals, particularly mammals, where the physiological sources from multicellular organisms may be derived from particular organs or tissues of the multicellular organism, or from isolated cells or subcellular/extracellular fractions derived therefrom.

As the subject methods are highly sensitive methods, in certain embodiments the subject methods are capable of detecting the presence, including quantitating the presence, of an extremely small copy number of target nucleic acid in a sample, where the copy number may be as low as 1000, including as low as 100, e.g., as low as 10, in a given sample having volume of at least about 5 microliters. As indicated above, the subject methods can be used to assay simple or complex samples, where complex samples are samples that include at least about 100 different nucleic acids of differing sequence. In obtaining the sample to be analyzed from the physiological source from which it is derived, the physiological source may be subjected to a number of different processing steps, where such processing steps might include tissue homogenization, nucleic acid extraction and the like, where such processing steps are known to the those of skill in the art. Methods of isolating nucleic acids, including RNA, from cells, tissues, organs or whole organisms are known to those of skill in the art. Representative such methods include those described in Maniatis et al., Molecular Cloning: A Laboratory Manual (Cold Spring Harbor Press)(1989).

In one embodiment, the sample is pretreated by one or more different protocols in order to reduce interference that may be caused by the presence of non-target nucleic acids, e.g., DNA and RNA, in the sample. For example, the sample may be contacted with a phosphatase enzyme, as exemplified in the experimental section below. In certain embodiments, the phosphatase is a heat-labile phosphatase, such as HK phosphatase available from Epicentre, shrimp phosphatase, etc. Use of heat labile phosphatases may be desirable in that such may be readily inactivated prior to practice of the remaining steps of the methods. In addition, non-heat labile phophatases are known in the art that may be easily removed after treatment, and therefore also may be used. Another representative pretreatment protocol is the removal of potentially interfering nucleic acids from the sample by size, leaving the shorter target nucleic acids in the sample and removing longer potentially interfering nucleic acids. To reduce interference from endogenous RNA, protocols for eliminating large RNAs are well known in the art. For example, extraction procedures using glass particles can selectively eliminate larger polynucleotides. Also known are memembrane filtration protocols. Procedures for subtracting polyadenylated mRNA are well established, using oligo-dT column, beads, and related products.

Where the target nucleic acid is siRNA, the sample may be pretreated to produce a surrogate target that is dependent upon the target siRNA and that is readily detectable by the subject methods. As is known in the art, one method for introducing siRNA into cells is the expression from DNA of single RNA molecules that contains both sense and antisense sequences. Such polynucleotides are capable of base-pairing between these sequences to form a “hairpin” structure, called shRNA. The shRNA may be processed by the cell into a double-stranded siRNA with some or the entire hairpin removed. The present invention measures siRNA directly.

The hairpin shRNA base pairs with itself and, therefore, may compete strongly with the binding of the DNA ligation domains used for ligation, as described in greater detail below. Annealing conditions that favor RNA-DNA heteroduplex formation over RNA-RNA binding are known in the art and may be employed in order to ensure that the single stranded RNA molecule does not hybridize with itself to produce a shRNA, but instead hybridizes with the two or more ligation domains. However, in certain embodiments, the shRNA is converted to siRNA or at least a structure with a gap between the complementary sequences with nucleases, so that the target nucleic acid is a double stranded RNA complex. A limited S1 nuclease treatment cuts the single-stranded loop of the hairpin without degrading the complementary sequences. Other nucleases are preferentially capable of cutting single-stranded RNA over double-stranded molecules. After or during inactivation or removal of the nuclease, the gapped molecule is heat treated to separate the strands. The shRNA is thereby converted into siRNA for purposes of this invention.

In another embodiment of converting shRNA to siRNA, RNAseH and a DNA oligonucleotide that binds within the open loop of the shRNA are employed. Upon formation of the RNA-DNA heteroduplex, RNAseH degrades specifically the RNA portion, thereby liberating the DNA to bind to another shRNA molecule. In this manner, shRNA is catalytically and specifically converted to siRNA or a functional equivalent. High temperature is used to inactivate the RNAseH and to separate the strands of the newly created siRNA

In another embodiment, the binding of a single-stranded DNA molecule or other nucleic acid forms to a portion of the target RNA sequence (sense or antisense) and to some or all of the open loop of shRNA is employed. This binding strongly inhibits the annealing of the sense and antisense sequences in the shRNA. The binding of the DNA to the loop sequences greatly increases the affinity and avidity of the DNA-RNA heteroduplex formation. The DNA sequence used to open up the hairpin does not cover the entire target RNA sequence, so that the DNA is not an efficient scaffold for the oligonucleotides used for ligation.

In practicing the subject methods, the first step is to produce a reaction mixture by contacting the sample to be assayed with at least two different ligation domains, which domains may be present on the same nucleic acid or on two or more different nucleic acids, e.g., oligonucleotides. Contact of the sample and the at least two different ligation domains may be achieved using any convenient protocol, e.g., combining the components into the same reaction vessel from different vessels, adding the sample to a vessel that include the ligation domains, adding the ligation domains to a vessel that contains the sample, etc.

In certain embodiments, the sample is contacted with more than two ligation domains, e.g., present as more than two different oligonucleotides, e.g., three or more, four or more etc., while in other embodiments, the sample is contacted with just two ligation domains. The ligation domains are characterized by including target nucleic acid hybridization domains (i.e. target nucleic acid complementary domains) that hybridize under stringent conditions to different portions/domains or regions of the target nucleic acid for which they are designed. As such, the sample is contacted with a set of two or more ligation domains for each different target nucleic acid to be detected, where a given set of ligation domains may include two or more different ligation oligonulceotides, each of which includes a different target nucleic acid hybridization domain, or may include single nucleic acid that includes two different ligation domains. In certain embodiments, the target nucleic acid hybridization domains of the ligation domains are designed or chosen so that when the ligation domains are hybridized to the target nucleic acid, there is no gap or space of one or more nucleotide residues on the target nucleic acid that is not hybridized to the juxtaposed ligation domains. In other words, the hybridization domains of the ligation domains are designed to hybridize to immediately adjacent regions or domains of the target nucleic acid that are not separated from each other by one or more nucleotides. In yet other embodiments, a space of one or more nucleotides may be present, which space is generally filled in, e.g., via use of a polymerase, prior to the next ligation step of the subject methods.

The target nucleic acid hybridization domains of the ligation domains may range in length, but in many embodiments typically range in length from about 5 to about 20 nt, such as from about 5 to about 15 nt, including from about 5 to about 13 nt.

Depending on the particular detection protocol employed in the subject methods (as described in greater detail below), in many embodiments the ligation domains may include, in addition to the target nucleic acid hybridization domain, a PCR primer domain. The PCR primer domain is a domain that is capable of serving as a primer binding site for a primer oligonucleotide in a PCR reaction, and may have any convenient nucleic acid sequence.

As indicated above, the ligation domains employed in the subject methods may be present on distinct nucleic acids, e.g., oligonucleotides, or present on the same nucleic acid. As such, in certain embodiments, each distinct ligation domain is present on a different nucleic acid, e.g., oligonucleotide, such that the method employs two or more (i.e., at least two) different ligation oligonucleotides. The ligations oligonucleotides employed in these embodiments are sufficiently long to at least include the ligation domains, and any other desired domains, e.g., PCR amplification domains, etc.

In another embodiment of this invention, the ligation domains are present on the same complex, e.g., nucleic acid, where two different ligation domains are connected to each other, either covalently or non-covalently, to form a single molecule or complex (also referred to herein as a “Combined Oligo”) with ends that are capable of annealing to the target nucleotide (e.g., siRNA) and being ligated together. After the ligation event, described in greater detail below, a circular molecule or complex (“Circular Oligo”) is produced.

In these embodiments, typically the two different ligation domains are separated from each other by a spacer region. The spacer region between the ligation domains may have any chemical structure that does not interfere with the annealing and ligation of the Combined Oligo to the target nucleotide. The spacer region is desirably of sufficient length to prevent steric hindrance of the Combined Oligo annealing to the target nucleic acid. The spacer region may include nucleotide sequences or chemical structures that allow for the detection of the Circular Oligo (by PCR, branch DNA, or rolling circle amplification, for example).

In one representative embodiment the spacer region is entirely composed of nucleotides, such that the terminal ligation domains, i.e., domain A and domain B, are present on a single nucleic acid. The spacer region may contain additional sequences that are amplifiable by PCR or other methods.

The Combined Oligo can also exist as a stable complex of two distinct oligonucleotides, one for each ligation domain, that are non-covalently attached. For example, a first oligonucleotide can contain biotin, and a second oligonucleotide can contain streptavidin. When the two reagents are mixed together, a complex forms as a result of high-affinity binding between biotin and streptavidin. This complex is virtually as stable as a single covalent molecule containing both the first and second oligonucleotides. Many non-covalent linkers can be used to make a functionally equivalent complex.

The Combined Oligo can also be made up first and second oligonucleotides that base pair to form a partially double-stranded complex. For example, the 5′ end of the first oligonucleotide and the 3′ end of the second nucleic acid may be constructed to have complementary DNA sequences, such that under the hybridization and ligation conditions of this invention these ends remain a stable DNA pairing. One skilled in the art can easily select such sequences to have appropriate annealing temperatures based on AT and GC composition. By mixing these two oligos together in equal molarities at a temperature above the annealing temperature and slowly cooling, a complex that includes both the first and second oligonucleotides is created that is a Combined Oligo with non-covalent binding between the first and second oligonucleotides. An advantage of this type of Combined Oligo is that after the ligation event, the Circular Oligo can be “melted” apart by high temperature to form a linear oligonucleotide that is easily amplifiable by PCR and other methods.

In yet other representative embodiments, Combined Oligos with covalent and non-covalent bonds connecting the first and second oligonucleotides in multiple different formats are employed. These Combined Oligos are functionally equivalent in allowing (1) the annealing of a single molecule or complex to the target nucleic acid, (2) the ligation of the first and second ligation domains, and (3) amplification of the pseudotarget nucleic acid produced by the ligation. The Combined Oligo is used virtually in the same manner as the separated ligation oligos described earlier.

Contact of the sample at least suspected of having the target nucleic acid and the ligation domains to produce a reaction mixture, as described above, is done in a manner sufficient to produce a complex between the ligation domains and a single strand of the target nucleic acid. In other words, this step is carried out in a manner that produces a complex between a single stranded target nucleic acid and at least two ligation domains, e.g., two ligation oligonucleotides, a Combined Oligo, etc. Accordingly, this step may be characterized as an annealing step, in which two or more ligation domains, such as two ligation oligonucleotides or a Combined Oligo, are contacted with a single stranded target nucleic acid to produce a complex of a target nucleic acid hybridized to the two or more ligation domains.

In those embodiments where the target nucleic acid is one that is initially double stranded, this annealing step typically also includes a dissociation step so that the double stranded target nucleic acid dissociates into single stranded nucleic acids which then anneal to the ligation oligonucleotides. However, strand migration and reaction conditions that favor heteroduplex formation will be sufficient in certain cases to allow the ligation domains to anneal to the target sequences without a separate dissociation step. As such, in these embodiments the ligation domains, e.g., olignucleotides, are contacted with the sample at least suspected of containing the target nucleic acid in an aqueous medium to produce a reaction mixture which is maintained under conditions sufficient for the target nucleic acid hybridization domains of the ligation domains/oligonucleotides to hybridize to their complementary sequences in the target nucleic acid, if present, in the sample. Before this annealing step of the embodiments where the target nucleic acid is initially double-stranded, the strands of the target nucleic acid are typically disassociated and then allowed to anneal in the presence of the ligation domains/oligonucleotides. In this annealing step, the ligation domains/oligonucleotides may be contacted with the sample before or after the sample has been subjected to strand disassociation conditions. The temperature of the reaction mixture is then reduced so that complementary strands in the reaction mixture re-associate, and target nucleic acid hybridization domains of the ligation domains/oligonucleotides hybridize to their complementary sequences present in the reaction mixture.

As such, in the annealing step of the subject methods, the sample may be first subjected to strand disassociation conditions, e.g., subjected to a temperature ranging from about 50° C. to about 100° C., usually from about 90° C. to about 95° C. for a period of time, and the resultant disassociated molecules then contacted with the ligation domain/oligonucleotide molecule(s) under annealing conditions, where the temperature of the composition is reduced to an annealing temperature of from about 20° C. to about 80° C., usually from about 25° C. to about 45° C. In certain embodiments, a “snap-cooling” protocol is employed, where the temperature is reduced to the annealing temperature, or to about 4° C. or below in a period of from about 1 to about 30 sec, usually from about 5 to about 10 sec.

As indicated above, the annealing step results in the production of ligation domain/target nucleic acid complexes, where the complexes are characterized by having target hybridization domains of ligation domains, e.g., oligonucleotides or Combined Oligo, hybridized to complementary domains of a target nucleic acid strand. In certain embodiments where the ligation domains do not hybridize to immediately adjacent domains of the target nucleic acid strand such that a gap remains on the target nucleic acid strand between the any two adjacent hybridized domains that has one or more nucleotide residues not hybridized to a residue of a complementary nucleic acid. The gap in these embodiments may be one or more nt in length, e.g., 5 or more, 10 or more, 15 or more, 20 or more, etc., nt in length. In these particular embodiments, the resultant gap is “filled in” using any convenient protocol, e.g., with a polymerase and the requisite nucleotides, to produce a structure in which no gap (as described above) exists on the target nucleic acid of non-hybridized residues to adjacent ligation domains/oligonucleotides.

The above annealing step (and any “filling in” step as desired) results in the production of double stranded nucleic acid complexes that include a strand of the target nucleic acid hybridized to immediately adjacent ligation domains/oligonucleotides, where the hybridized ligation domains/oligonucleotides are not separated by each other by any missing nucleotides. In many embodiments, e.g., where detection is by PCR, the resultant complex may further include single stranded domains at either end of the duplex structure, which domains comprise the PCR primer domains.

In certain embodiments, the above annealing step includes the use of one or more different reagents that improve the fidelity of the annealing of the ligation domains/oligonucleotides to the target nucleic acid. Many reagents are known to increase the fidelity of annealing and, therefore, may serve to decrease unwanted background (non-specific) annealing and consequent ligation events. Solvents, such as glycerol, DMSO, and formamide, are reported to increase the fidelity of PCR reactions by increasing the stringency of the binding of primers to the target DNA. Biopolymers, such as T4 Gene 32 protein, are also known to increase the specificity of annealing. Such reagents may be used to increase the fidelity of binding of DNA to the RNA target and, therefore, reduce the background of non-specific ligations.

Tetraalkylammonium chlorides and related compounds are particularly useful for increasing the fidelity of ligation, as well as the subsequent PCR reactions (Hung, et al., 1990, Nucleic Acid Res. 18:4953). Tetraethylammonium chloride (TEAC) and tetramethylammonium chloride (TMAC) have been used in polynucleotide hybridization protocols to increase the stringency of binding and, therefore, to decrease non-specific, background annealing events. Furthermore, these compounds have an effect on base-pairing that results in a more uniform binding of CG (Watson-Crick) pairs compared to AT pairs in DNA-DNA interactions. In RNA-RNA or RNA-DNA binding, TEAC and TMAC are known to make more uniform the binding of CG compared to AU. This normalization of binding energies leads to annealing kinetics that are more dependent upon the length of the polynucleotides, instead on a specific sequence.

In the next step of the subject methods, the adjacent hybridized ligation domains/olignucleotides of the above described complexes are ligated to each other to produce a pseudotarget nucleic acid, i.e., a secondary surrogate structure that is characterized by including a least a portion of the full-length sequence of the target nucleic acid.

In many embodiments of the subject invention, the adjacent ligation oligonucleotides are ligated to each other in this ligation step by using a ligase. As is known in the art, ligases catalyze the formation of a phosphodiester bond between juxtaposed 3′-hydroxyl and 5′-phosphate termini of two immediately adjacent oligonucleotides when they are annealed or hybridized to a third nucleic acid sequence to which they are complementary. Any convenient ligase may be employed, where representative ligases of interest include, but are not limited to Temperature sensitive and thermostable ligases. Temperature sensitive ligases, include, but are not limited to, bacteriophage T4 DNA ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eucaryotic, or archael organisms. Certain RNA ligases may also be employed in the methods of the invention. In this ligation step, a suitable ligase and any reagents that are necessary and/or desirable are combined with the reaction mixture and maintained under conditions sufficient for ligation of the hybridized ligation oligonucleotides to occur. Ligation reaction conditions are well known to those of skill in the art. During ligation, the reaction mixture in certain embodiments may be maintained at a temperature ranging from about 20° C. to about 45° C., such as from about 25° C. to about 37° C. for a period of time ranging from about 5 minutes to about 16 hours, such as from about 1 hour to about 4 hours. In yet other embodiments, the reaction mixture may be maintained at a temperature ranging from about 35° C. to about 45° C., such as from about 37° C. to about 42° C., e.g., at or about 38° C., 39° C., 40° C. or 41° C., for a period of time ranging from about 5 minutes to about 16 hours, such as from about 1 hour to about 10 hours, including from about 2 to about 8 hours. In representative embodiments, the ligation oligo concentration may range from between about 10 nM and about 500 nM for each oligo. In a representative embodiment, the ligation reaction mixture includes 50 mM Tris pH7.5, 10 mM MgCl₂, 10 mM DTT, 1 mM ATP, 25 μg/ml BSA, 0.25 units/μl Rnase inhibitor, and T4 DNA ligase at 0.125 units/μl. In yet another representative embodiment, 2.125 mM magnesium ion, 0.2 units/μl Rnase inhibitor; and 0.125 units/μl DNA ligase are employed.

The above described ligation step results in the production of a pseudotarget nucleic acid, which nucleic acid is the product of ligation of the two or more ligation oligonucleotides, as described above. As indicated above, in certain embodiments, the pseudotarget nucleic acid, in addition to including a sequence complementary to at least a significant portion of, if not all of the target nucleic acid, may further include 5′ and/3′ additional domains, which domains may range in length from about 2 to about 100 nt, including from about 5 to about 50 nt. In certain embodiments, the pseudotarget or surrogate target nucleic acid is one that is longer than the target nucleic acid, e.g., by at least about 5 nt, at least about 10 nt, at least about 20 nt, at least about 50 nt, or more.

At any convenient time during the above portions of the subject methods, a nuclease inhibitor may be-included in the reaction mixture, e.g., an RNA nuclease inhibitor. For example, where the target nucleic acid is an siRNA, the siRNA is vulnerable to degradation during the RNA extraction process, storage, and template-dependent ligation. Protocols to reduce nuclease contamination and products that inhibit nucleases are well known and are incorporated in the present invention. Placental RNAse inhibitor and vanadyl ribonucleotide complex are two examples of commercially available inhibitors of RNAse.

The next step of the subject methods following the above-described ligation step is to determine the presence of the pseudotarget nucleic acid in the reaction mixture in order to detect the target nucleic acid in the sample. In other words, the reaction mixture is screened, i.e., assayed, evaluated, tested, etc., for the presence of any resultant ligation products in order to detect the presence of the target nucleic acid in the sample being assayed.

The product pseudotarget nucleic acid may, in the broadest sense, be detected using any convenient protocol. The particular detection protocol may vary depending on the sensitivity desired and the application in which the method is being practiced, as well as the particular nature of the pseudotarget nucleic acid, e.g., whether it is a linear or circular molecule/complex. In certain embodiments, the product pseudotarget nucleic acid is directly detected without any amplification, while in other embodiments the detection protocol may include an amplification component, in which the copy number of the product pseudotarget nucleic acid is increased, e.g., to enhance sensitivity of the particular assay. Where detection without amplification is practicable, the product pseudotarget nucleic acid may be detected in a number of different ways. For example, one or more of the ligation oligonucleotides may have been directly labeled, e.g., fluorescently or radioisotopically labeled, such that the ligation product is directly labeled. In these embodiments, the directly labeled ligation product may be size separated from the remainder of the reaction mixture, including unligated directly labeled ligation oligonucleotides, in order to detect the pseudotarget nucleic acid. Alternatively, the ligation oligonucleotides may each include fluorescent moieties which give rise to different signals depending on whether or not they are present in the same ligation product or as separate ligation oligonucleotides, e.g., they may be labeled with FRET pairs, as described in greater detail below. In yet other non-amplification embodiments, conformationally selective probes, e.g., molecular beacons (as described in greater detail below) may be employed to detect to the presence of the pseudotarget nucleic acid, where these probes are directed to a sequence that spans the ligated oligonucleotides and therefore only exists in its entirety in the ligation product.

As indicated above, in certain embodiments of the subject methods, the detection step includes an amplification step, where the copy number of pseudotarget nucleic acids is increased, e.g., in order to enhance sensitivity of the assay. The amplification may be linear or exponential, as desired, where representative amplificaiton protocols of interest include, but are not limited to: polymerase chain reaction (PCR); rolling circle amplification; isothermal amplification, branched DNA, etc.

The polymerase chain reaction (PCR), is well known in the art, being described in U.S. Pat. Nos. 4,683,202; 4,683,195; 4,800,159; 4,965,188 and 5,512,462, the disclosures of which are herein incorporated by reference. In representative PCR amplification reactions, the reaction mixture that includes the above pseudotarget nucleic acid or ligation product (which may also be viewed as a template nucleic acid in an amplification reaction) is combined with one or more primers that are employed in the primer extension reaction, e.g., the PCR primers (such as forward and reverse primers employed in geometric (or exponential) amplification or a single primer employed in a linear amplification). The oligonucleotide primers with which the template nucleic acid (hereinafter referred to as template DNA for convenience) is contacted will be of sufficient length to provide for hybridization to complementary template DNA under annealing conditions (described in greater detail below. The primers will generally be at least 10 bp in length, usually at least 15 bp in length and more usually at least 16 bp in length and may be as long as 30 bp in length or longer, where the length of the primers will generally range from 18 to 50 bp in length, usually from about 20 to 35 bp in length. The template DNA may be contacted with a single primer or a set of two primers (forward and reverse primers), depending on whether primer extension, linear or exponential amplification of the template DNA is desired.

In addition to the above components, the reaction mixture produced in the subject methods typically includes a polymerase and deoxyribonucleoside triphosphates (dNTPs). The desired polymerase activity may be provided by one or more distinct polymerase enzymes. In many embodiments, the reaction mixture includes at least a Family A polymerase, where representative Family A polymerases of interest include, but are not limited to: Thermus aquaticus polymerases, including the naturally occurring polymerase (Taq) and derivatives and homologues thereof, such as Klentaq (as described in Proc. Natl. Acad. Sci USA (1994) 91:2216-2220); Thermus thermophilus polymerases, including the naturally occurring polymerase (Tth) and derivatives and homologues thereof, and the like. In certain embodiments where the amplification reaction that is carried out is a high fidelity reaction, the reaction mixture may further include a polymerase enzyme having 3′→5′ exonuclease activity, e.g., as may be provided by a Family B polymerase, where Family B polymerases of interest include, but are not limited to: Thermococcus litoralis DNA polymerase (Vent) as described in Perler et al., Proc. Natl. Acad. Sci. USA (1992) 89:5577; Pyrococcus species GB-D (Deep Vent); Pyrococcus furiosus DNA polymerase (Pfu) as described in Lundberg et al., Gene (1991) 108:1-6, Pyrococcus woesei (Pwo) and the like. Where the reaction mixture includes both a Family A and Family B polymerase, the Family A polymerase may be present in the reaction mixture in an amount greater than the Family B polymerase, where the difference in activity will usually be at least 10-fold, and more usually at least about 100-fold. Usually the reaction mixture will include four different types of dNTPs corresponding to the four naturally occurring bases are present, i.e. dATP, dTTP, dCTP and dGTP. In the subject methods, each dNTP will typically be present in an amount ranging from about 10 to 5000 uM, usually from about 20 to 1000 uM.

The reaction mixture prepared in this detection step of the subject methods may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulfate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 3 to 6 mM, and will ideally be at about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, where most preferred is pH 7.3 at 72° C. Other agents which may be present in the buffer medium include chelating agents, such as EDTA, EGTA and the like.

If PCR is employed to detect a pseudotarget nucleic acid that is produced from a Combined Oligo, the protocol may be developed such that the PCR product spans across the newly ligated region of the Circular Oligo in the pseudotarget nucleic acid product. In the absence of ligation of the Combined Oligo, amplification occurs only as a minor contaminating reaction (e.g., primer dimer amplification). To prevent continuous production of concatamers from the pseudotarget nucleic acid, the spacer region can consist of chemical moieties that are non-nucleotides or nucleotides that prevent elongation by polymerases (e.g., inverted dT). Persons skilled in the art can easily select moieties for the spacer region that connect the ligation oligo sequences through a covalently joined spacer, wherein the spacer contains moieties that prevent or restrict procession of a polymerase across the spacer.

If branched DNA is used for detecting the ligation event in a Combined Oligo, the detection probe binds strongly to the newly formed junction of the pseudotarget nucleic acid and not to the unligated Combined Oligo. Those skilled in the art can easily create probes that efficiently anneal across the ligation junction and that virtually show no binding to the unligated starting material under specific temperature and buffer conditions, because the unligated Combined Oligo will have significantly fewer continuous sequences with which to base pair to the probe.

In yet other embodiments, e.g., where a Combined Oligo is employed to produce the pseudotarget nucleic acid, a rolling circle amplification protocol may be employed. In these rolling circle amplification protocols, the pseudotarget nucleic acid is a circular nucleic acid that serves as a template for geometric rolling circle amplification, in which forward and reverse rolling circle primers are contacted with the circular template under rolling circle amplification conditions sufficient to produce long complementary DNA strands that, upon hybridization to each other, include multiple copies of the target nucleic acid sequence or complement thereof. Rolling circle amplification conditions are known in the art and described in, among other locations, U.S. Pat. Nos. 6,576,448; 6,287,824; 6,235,502; and 6,221,603; the disclosures of which are herein incorporated by reference.

In preparing the reaction mixture of this step of the subject methods, the various constituent components may be combined in any convenient order. For example, the buffer may be combined with primer, polymerase and then template DNA, or all of the various constituent components may be combined at the same time to produce the reaction mixture.

The amplified products of the amplification reaction may be detected using any convenient protocol, where the particular protocol employed may detect the amplification products non-specifically or specifically, as described in greater detail below. Representative non-specific detection protocols of interest include protocols that employ signal producing systems that selectively detect double stranded DNA products, e.g., via intercalation. Representative detectable molecules that find use in such embodiments include fluorescent nucleic acid stains, such as phenanthridinium dyes, including monomers or homo- or heterodimers thereof, that give an enhanced fluorescence when complexed with nucleic acids. Examples of phenanthridinium dyes include ethidium homodimer, ethidium bromide, propidium iodide, and other alkyl-substituted phenanthridinium dyes. In another embodiment of the invention, the nucleic acid stain is or incorporates an acridine dye, or a homo- or heterodimer thereof, such as acridine orange, acridine homodimer, ethidium-acridine heterodimer, or 9-amino-6-chloro-2-methoxyacridine. In yet another embodiment of the invention, the nucleic acid stain is an indole or imidazole dye, such as Hoechst 33258, Hoechst 33342, Hoechst 34580 (BIOPROBES 34, Molecular Probes, Inc. Eugene, Oreg., (May 2000)) DAPI (4′,6-diamidino-2-phenylindole) or DIPI (4′,6-(diimidazolin-2-yl)-2-phenylindole). Other permitted nucleic acid stains include, but are not limited to, 7-aminoactinomycin D, hydroxystilbamidine, LDS 751, selected psoralens (furocoumarins), styryl dyes, metal complexes such as ruthenium complexes, and transition metal complexes (incorporating Tb³⁺ and Eu³⁺, for example). In certain embodiments of the invention, the nucleic acid stain is a cyanine dye or a homo- or heterodimer of a cyanine dye that gives an enhanced fluorescence when associated with nucleic acids. Any of the dyes described in U.S. Pat. No. 4,883,867 to Lee (1989), U.S. Pat. No. 5,582,977 to Yue et al. (1996), U.S. Pat. No. 5,321,130 to Yue et al. (1994), and U.S. Pat. No. 5,410,030 to Yue et al. (1995) (all four patents incorporated by reference) may be used, including nucleic acid stains commercially available under the trademarks TOTO, BOBO, POPO, YOYO, TO-PRO, BO-PRO, PO-PRO and YO-PRO from Molecular Probes, Inc., Eugene, Oreg. Any of the dyes described in U.S. Pat. No. 5,436,134 to Haugland et al. (1995), U.S. Pat. No. 5,658,751 to Yue et al. (1997), and U.S. Pat. No. 5,863,753 to Haugland et al. (1999) (all three patents incorporated by reference) may be used, including nucleic acid stains commercially available under the trademarks SYBR, SYTO, SYTOX, PICOGREEN, OLIGREEN, and RIBOGREEN from Molecular Probes, Inc., Eugene, Oreg. In yet other embodiments of the invention, the nucleic acid stain is a monomeric, homodimeric or heterodimeric cyanine dye that incorporates an aza- or polyazabenzazolium heterocycle, such as an azabenzoxazole, azabenzimidazole, or azabenzothiazole, that gives an enhanced fluorescence when associated with nucleic acids, including nucleic acid stains commercially available under the trademarks SYTO, SYTOX, JOJO, JO-PRO, LOLO, LO-PRO from Molecular Probes, Inc., Eugene, Oreg.

In yet other embodiments, a signal producing system that is specific for the amplification product, as opposed to double stranded molecules in general, may be employed to detect the amplification. In these embodiments, the signal producing system may include a probe nucleic acid that specific binds to a sequence found in the amplification product, where the probe nucleic acid may be labeled with a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, where directly detectable labels of interest include, but are not limited to: fluorescent labels, radioisotopic labels, chemiluminescent labels, and the like. In many embodiments, the label is a fluorescent label, where the labeling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels, such as those described above, may also be employed as are known in the art.

In certain embodiments, the specifically labeled probe nucleic acids are labeled with “energy transfer” labels. As used herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena. As used herein, “energy transfer pair” refers to any two molecules that participate in energy transfer. Typically, one of the molecules acts as a fluorescent group, and the other acts as a fluorescence-modifying group. “Energy transfer pair” is used to refer to a group of molecules that form a single complex within which energy transfer occurs. Such complexes may comprise, for example, two fluorescent groups which may be different from one another and one quenching group, two quenching groups and one fluorescent group, or multiple fluorescent groups and multiple quenching groups. In cases where there are multiple fluorescent groups and/or multiple quenching groups, the individual groups may be different from one another. As used herein, “fluorescence resonance energy transfer” or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly. As used herein “direct energy transfer” refers to an energy transfer mechanism in which passage of a photon between the fluorescent group and the fluorescence-modifying group does not occur. Without being bound by a single mechanism, it is believed that in direct energy transfer, the fluorescent group and the fluorescence-modifying group interfere with each others electronic structure. If the fluorescence-modifying group is a quenching group, this will result in the quenching group preventing the fluorescent group from even emitting light.

The energy transfer labeled probe nucleic acid, e.g., oligonucleotide, may be structured in a variety of different ways, so long as it includes a donor, acceptor and target nucleic acid binding domains. As such, the energy transfer labeled oligonucleotides employed in these embodiments of subject methods are nucleic acid detectors that include a fluorophore domain where the fluorescent energy donor, i.e., donor, is positioned and an acceptor domain where the fluorescent energy acceptor, i.e., acceptor, is positioned. As mentioned above, the donor domain includes the donor fluorophore. The donor fluorophore may be positioned anywhere in the nucleic acid detector, but is typically present at the 5′ terminus of the detector. The acceptor domain includes the fluorescence energy acceptor. The acceptor may be positioned anywhere in the acceptor domain, but is typically present at the 3′ terminus of the nucleic acid detector or probe.

In addition to the fluorophore and acceptor domains, the energy transfer labeled probe oligonucleotides also include a target nucleic acid binding domain, which binds to a target nucleic acid sequence found in the amplification product of interest (as described above), e.g., under stringent hybridization conditions (as defined above). This target binding domain typically ranges in length from about 10 to about 60 nt, usually from about 15 to about 30 nt. Depending on the nature of the oligonucleotide and the assay itself, the target binding domain may hybridize to a region of the template nucleic acid or a region of the primer extension product. For example, where the assay is a 5′ nuclease assay, e.g., in which a Taqman™ type oligonucleotide probe is employed, the target binding domain hybridizes under stringent conditions to a target binding site of the template nucleic acid, which is downstream or 3′ of the primer binding site. In alternative embodiments, e.g., in molecular beacon type assays, the target binding domain hybridizes to a domain of a primer extension product. The overall length of the energy transfer labeled oligonucleotides employed in these embodiments, which includes all three domains mentioned above, typically ranges from about 10 to about 60 nt, usually from about 15 to about 30 nt.

In certain embodiments, the energy transfer labeled oligonucleotide is structured such that energy transfer occurs between the fluorophore and acceptor of the energy transfer labeled oligonucleotide probe upon fluorophore excitation when the energy transfer labeled oligonucleotide is not hybridized to target nucleic acid.

In certain embodiments, the oligonucleotide is a single stranded molecule that does not form intramolecular structures and in which energy transfer occurs because the spacing of the donor and acceptor provides for energy transfer in the single stranded linear format. In these embodiments, energy transfer also occurs between the fluorophore and acceptor of labeled oligonucleotide probe upon fluorophore excitation when the labeled oligonucleotide probe is hybridized to a target nucleic acid. Specific examples of such labeled oligonucleotide probes include the Taqman™ type probes, as described in U.S. Pat. No. 6,248,526, the disclosure of which is herein incorporated by reference (as well as Held et al., Genome Res. (1996) 6:986-994; Holland et al., Proc. Nat'l Acad. Sci. USA (1991) 88:7276-7280; and Lee et al., Nuc. Acids Res. (1993) 21:3761-3766 (1993)). In many of these embodiments, the target nucleic acid binding domain is one that hybridizes to, i.e., is complementary to, a sequence of the template nucleic acid, i.e., the target nucleic acid of the target nucleic acid binding domain is a sequence present in the template nucleic acid (i.e., the pseudotarget or surrogate nucleic acid).

In other embodiments, the probe oligonucleotides are structured such that energy transfer does not occur between the fluorophore and acceptor of the energy transfer labeled oligonucleotide probe upon fluorophore excitation when the energy transfer labeled oligonucleotide probe is hybridized to a target nucleic acid. Examples of these types of probe structures include: Scorpion probes (as described in Whitcombe et al., (Nature Biotechnology (1999) 17:804-807; U.S. Pat. No. 6,326,145, the disclosure of which is herein incorporated by reference), Sunrise probes (as described in Nazarenko et al., Nuc. Acids Res. (1997) 25:2516-2521; U.S. Pat. No. 6,117,635, the disclosure of which is herein incorporated by reference), Molecular Beacons (Tyagi et al., Nature Biotechnology (1996) 14:303-308; U.S. Pat. No. 5,989,823, the disclosure of which is incorporated herein by reference), and conformationally assisted probes (as described in provisional application Ser. No. 60/138,376, the disclosure of which is herein incorporated by reference). In many of these embodiments, the target binding sequence or domain comprises a hybridization domain complementary to a sequence of the primer extension product of the amplification reaction, and not to a sequence found in the pseudotarget nucleic acid.

The next step in the subject methods is signal detection from the labeled amplification products of interest, where signal detection may vary depending on the particular signal producing system employed. In certain embodiments, merely the presence or absence of detectable signal, e.g., fluorescence, is determined and used in the subject assays, e.g., to determine or identify the presence or absence of the target nucleic acid via detection of the pseudotarget nucleic acid and/or amplification products thereof. Depending on the particular label employed, detection of a signal may indicate the presence or absence of the target nucleic acid.

In those embodiments where the signal producing system is a fluorescent signal producing system, signal detection typically includes detecting a change in a fluorescent signal from the reaction mixture to obtain an assay result. In other words, any modulation in the fluorescent signal generated by the reaction mixture is assessed. The change may be an increase or decrease in fluorescence, depending on the nature of the label employed, but in certain embodiments is an increase in fluorescence. The sample may be screened for an increase in fluorescence using any convenient means, e.g., a suitable fluorimeter, such as a thermostable-cuvette or plate-reader fluorimeter. Fluorescence is suitably monitored using a known fluorimeter. The signals from these devices, for instance in the form of photo-multiplier voltages, are sent to a data processor board and converted into a spectrum associated with each sample tube. Multiple tubes, for example 96 tubes, can be assessed at the same time.

Where the detection protocol is a real time protocol, e.g., as employed in real time PCR reaction protocols, data may be collected in this way at frequent intervals, for example once every 10 ms, throughout the reaction. By monitoring the fluorescence of the reactive molecule from the sample during each cycle, the progress of the amplification reaction can be monitored in various ways. For example, the data provided by melting peaks can be analyzed, for example by calculating the area under the melting peaks and these data plotted against the number of cycles.

The spectra generated in this way can be resolved, for example, using “fits” of pre-selected fluorescent moieties such as dyes, to form peaks representative of each signaling moiety (i.e. fluorophore). The areas under the peaks can be determined which represents the intensity value for each signal, and if required, expressed as quotients of each other. The differential of signal intensities and/or ratios will allow changes in labeled probes to be recorded through the reaction or at different reaction conditions, such as temperatures. The changes are related to the binding phenomenon between the oligonucleotide probe and the target sequence or degradation of the oligonucleotide probe bound to the target sequence. The integral of the area under the differential peaks will allow intensity values for the label effects to be calculated.

Screening the mixture for a change in fluorescence provides one or more assay results, depending on whether the sample is screened once at the end of the primer extension reaction, or multiple times, e.g., after each cycle, of an amplification reaction (e.g., as is done in real time PCR monitoring).

The data generated as described above can be interpreted in various ways. In its simplest form, an increase or decrease in fluorescence from the sample in the course of or at the end of the amplification reaction is indicative of an increase in the amount of the target sequence present in the sample, e.g., as correlated to the amount of amplification product detected in the reaction mixture, suggestive of the fact that the amplification reaction has proceeded and therefore the target sequence was in fact present in the initial sample. Quantification is also possible by monitoring the amplification reaction throughout the amplification process. Quantification may also include assaying for one or more control nucleic acids in the reaction mixture, as described above.

In this manner, a reaction mixture is readily screened for the presence of target nucleic acids. The methods are suitable for detection of a single primer target nucleic acid as well as multiplex analyses, in which two or more different two or more different target nucleic acids are assayed in the sample. In these latter multiplex situations, the number of different types of probes that may be employed typically ranges from about 2 to about 20 or higher, usually from about 2 to about 15.

The above described methods of detecting the presence of one or more target nucleic acids in a complex sample finds use in a variety of different applications, representative ones of which are now reviewed in greater detail.

Utility

The above-describe methods find use in any application where the detection of one or more target nucleic acids in a sample, including a complex nucleic acid sample, is desired. Representative applications of interest include research and clinical, e.g., diagnostic and therapeutic, applications.

As such, one can use in the subject methods in research applications to rapidly identify the presence of one or more analyte nucleic acids of interest in a sample, where such applications could includes screening of various physiological samples to identify nucleic acids of interest, etc.

As indicated above, representative clinical applications of interest include both diagnostic and therapeutic applications. Accordingly, where a given host condition, e.g., disease condition, is characterized by the presence of one or more specific nucleic acids, the subject methods may be used to screen a sample from the host for the presence of the nucleic acids to thereby make a diagnosis of the presence of absence of the disease condition.

The subject methods also find use in therapeutic applications, e.g., in monitoring the progress of a therapeutic protocol, or as part of the protocol itself. For example, where the progress of a given therapeutic protocol for a disease condition may be assessed or evaluated based on the presence and/or amount of one or more target nucleic acids present in a sample from the host, a sample from the host may be assayed using the subject methods to monitor the disease condition.

In yet other embodiments, the subject methods may be incorporated into a therapeutic treatment regimen per se. For example, in siRNA agent treatment protocols where an siRNA agent is administered to a group of cells harvested from a patient, where the harvested cells are expanded ex vivo and then readministered to the patient, the subject methods may be used to identify those cells that include a sufficient amount of the RNAi therapeutic agent, and therefore those cells that should be expanded and readministered to the host.

A representative protocol for ex vivo transfection of an isolated population of cells for later readministration into a patient in which the subject methods are employed may include the following steps:

(a) isolating the desired population of a patient's cells from the patient;

(b) transducing the isolated population of cells with an siRNA or antisense construct;

(c) determining the level of the siRNA or antisense construct in and outside the cells for a target antisense or siRNA construct, where the target is a single stranded or double stranded polynucleotide having a 3′ end and a 5′ end and having a sense strand and an antisense strand if double-stranded or an antisense strand if single stranded, by a process comprising:

(i) generating two oligonucleotides corresponding to and extending beyond the 3′ end (oligonucleotide A) and the 5′ end (oligonucleotide B) of the target polynucleotide;

(ii) annealing the two oligonucleotides to the polynucleotide antisense or sense strand to form a partial double stranded polynucleotide;

(iii) ligating the partial double stranded polynucleotide to form a target surrogate;

(iv) amplifying the target surrogate; and

(v) analyzing the quantity of the target surrogate to quantify the presence of the target single stranded or double stranded polynucleotide;

(d) expanding the cell population having appropriate level of transduction to form an expanded transduced cell population; and

(e) re-administering the expanded transduced cell population to the patient.

Further examples of where the subject methods find use include those specific protocols described in application Nos. 60/488,486 and 60/491,910; the disclosures of which are herein incorporated by reference.

Systems

Also provided are systems for practicing one or more of the above-described methods. The systems typically at least include at least two ligation domains, e.g., in the form of a Combined Oligo or two or more ligation oligonucleotides, complementary to different regions of the target nucleic acid; a ligase; and pseudotarget detection reagents, e.g., which may or may not include amplification reagents, as described above. The system may also include a labeled product detection device, e.g., a fluorimeter, etc.

Kits

Also provided are reagents and kits thereof for practicing one or more of the above-described methods. The subject reagents and kits thereof may vary greatly. In certain embodiments, the kits include at two or more ligation domains, e.g., the in form of a Combined Oligo or in the form of two or more ligation oligonucleotides, as described above. In certain embodiments, the kits may further include one or more additional reagents that find use in practicing the subject methods, e.g., a ligase, pseudotarget detection reagents, including amplification reagents, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### EXAMPLE 1

### Quantification of siRNA in 293T Cells

Plasmid pTZ U6+1 (from the City of Hope) was used as a DNA vector for transfection of DNA sequences that were employed to generate siRNAs inside 293T cells. DNA sequences corresponding to the siRNAs were cloned into pTZ U6+1 downstream from the U6 promoter. The plasmids were, therefore, capable of expressing siRNA from the U6 promoter when transfected into the 293T cells. The plasmids contained a bacterial origin of replication (from pUC background) and an ampicillin-resistance gene. The plasmids with siRNA DNA sequences were replicated in E. coli, using drug selection and standard methods, and are purified with Endo-free Maxi Kit (Qiagen).

In one embodiment, two different plasmids were introduced into 293T cells, one encoding a sense stand of the siRNA (tet/rev1+) and other encoding the antisense strand of the siRNA (tet/rev1−). The sequences of tet/rev1+ and tet/rev1− are provided below:

| | |
|---|---|
| Oligo ID | Sequence |

| | | | |
|---|---|---|---|
| tet/rev1+ | GCGGAGACAGCGACGAAGAGC | (sense sequence) | (SEQ ID NO:01) |
| tet/rev1− | GCUCUUCGUCGCUGUCUCCGC | (antisense sequence) | (SEQ ID NO:02) |

In another embodiment, a single plasmid encoding the following shRNA molecule was introduced into 293T cells.

| | |
|---|---|
| siRNA1 Hairpin | |
| GCGGAGACAGCGACGAAGAGCUUCCCCUCGCUCUUCGUCGCUGUCUCCGC | (SEQ ID NO:03) |

Lipofectamine (Invitrogen) was used for transfecting the purified plasmids into 293T cells. Cells were treated with the transfection reagents and were placed in Opti-MEM I medium and later Opti-MEM I medium with fetal bovine serum. After one day the cells were grown on DMEM medium. Cells were harvested after two or more days to determine siRNA levels.

A sample of total RNA was isolated from about 1×10⁷ cells by first suspending the cells in culture medium with a scraper (293T cells being an attached cell line). The cell suspension was centrifuged at 250g for 15 min at room temp. The supernatant was completely removed; and the cell pellet was lysed by adding 1 ml of TRIZOL reagent (Invitrogen).

Following complete lysis of the cells, the resultant cell lysate was pipetted up and down repeatedly until no cell debris could be observed. To every 1 ml of cell lysate, 0.2 ml of chloroform was added. The proteins and DNA were denatured by vigorously shaking the tube for 1 min. The organic and aqueous phases were separated by centrifugation at 12,000 g at 4° C. for 15 min. There were three visible layers inside the tube. Total RNA was in the top aqueous phase, with denatured proteins and chromosome DNA in the whitish layer in between.

The top aqueous phase was carefully collected into a fresh tube without disturbing the denatured protein and DNA layer. About 600 μl of solution from the aqueous phase was usually collected from 1 ml of original lysate. Total RNA was precipitated by adding 0.5 ml of isopropanol. The precipitation mixture was incubated at room temperature for 10 min and precipitated at 12,000 g at 4° C. for 20 min. The RNA precipitate was usually a white-grayish color.

Based on the level of RNA expression from each cell, the size of the RNA pellet from different cells varied. The RNA precipitate was washed with 1 ml of 70% ethanol and very gently mixed by inverting the tube several times. The tube was returned back to centrifugation at 4° C. 12,000 g for 10 min. The ethanol was removed; and the RNA pellet air-dried at room temperature for 10-15 min.

Total RNA was dissolved in 77 μl of RNase-free H₂O and mixed with 9 μl of 10×DNase buffer and 4 U of RNase-free DNase (1 U/μl). The tube was incubated at room temperature for 15 min only. After incubation, the reaction was stopped by adding 10 μl of 25 mM EDTA solution and incubating at 65° C. for 10 min. Aliquots of RNA samples were immediately frozen at −70° C. for storage.

The foregoing total RNA samples were tested for the presence of siRNA primary target as follows. A total of 1 μl of RNA, 1 μl of each of two oligos (Oligo A having the sequence 5′-CGGTATTCGGAATCTTGCCATGGCCGGATCCGCGGAGACAGC-3′ (called Primer Tlig3c (SEQ ID NO. 4)) and oligo B having the sequence 5′-GACGAAGAGCTTTTTTTCTCTTACCAGCCTAACTT-3″ (called primer Tlig5c (SEQ ID NO. 5)) were mixed with 14 ml of RNase-free H₂O in a PCR tube. Using a thermo-cycler, the RNA and oligomer mixture was first denatured at 90° C. for 1 min and then gradually decreased to 30° C. at a ramp rate of 1° C./40 sec. The annealed mixture was kept at 30° C. for 30 min before adding 2 μl of 10×T4 ligation buffer and 1 μl of T4 DNA ligase without further lowering the temperature. The ligation reaction was completed in about 15 hours.

The secondary surrogate target created by the ligation of oligo A to oligo B using the primary target as a template was quantitatively analyzed by PCR using 1 μl of the ligated sample without further treatment.

Using the foregoing protocol, the process was first tested with pure synthesized single-stranded siRNAs. The siRNA-1 sense strand (8.732 nmol was dissolved in 87.3 μl of water to give a 100 μM solution (100 pmol/μl). It was further diluted 10-fold serially to 10 μM (10 pmol/μl), 1 μM (1 pmol/μl, 1×10¹¹ molecules/μl) and 0.1 μM (0.1 pmol/μl). Approximately 2 μl of the ligation mixture was used for PCR. In addition, the anti-sense strand of siRNA-1 (59.32 nmol total) was dissolved in 59.32 ml of water to give a final concentration of 1 mM (1 nmol/μl). It was 10-fold serially diluted to give 100 μM, 10 μM, 1 μM, 0.1 μM, 10 nM, 1 nM and 0.1 nM concentrations. Proper concentration curves were produced.

In another experiment, 1×10⁶ siRNA-1 plasmid transfected 293T cells were placed into each well of a 6-well plate. Two days after transfection, total RNA was isolated from every three wells and the total RNA was treated with DNase I. The secondary surrogate target was analyzed by real-time PCR using primer pair TL5 (CGGTATTCGGAATCTT (SEQ ID NO. 06)) and TL3 (AAGTTAGGCTGGTAAG (SEQ ID NO. 07)). The results showed the ability to quantify transfection and led to improvements in the transfection process for this plasmid. In addition, it was found that the sense strand of siRNA-1 was at least lower than 50 copies per cell. The anti-sense strand of siRNA-1 was at least lower than 100 copies per cell.

### EXAMPLE 2

### High Temperature Ligation Quantification Protocol

In this protocol, the target siRNA was:

| | |
|---|---|
| (5′GCUCUUCGUCGCUGUCUCCGC3′) | (SEQ ID NO:02) |

Ligation was performed with the following sequence altered oligonucleotides:

siRNA1.AS7 Lig5c:

| | |
|---|---|
| 5′CGGTATTCGGAATCTTGCCATGAGCGAGATGCAGCGGAGACAGC3′ | (SEQ ID NO:08) |
| and | |
| siRNA1.AS7 Lig3c: | |
| 5′/5Phos/GACGAAGAGCTCTTACCAGCCTAACTTAT3′. | (SEQ ID NO:09) |

In this protocol, each ligation oligonucleotide was at a final concentration of 4E⁻⁸ Molar. RNase inhibitor SUPERase-ln® was utilized at 0.2 Units/Microliter. T4DNA Ligase was utilized at 0.125 Units/Microliter. Ligations were incubated at 4° C. for 8 Hours.

PCR was performed as described previously above, but with primers TL5c and TL3c both at a final concentration of 0.3 Micromolar and Magnesium ion concentration adjusted to a final of 2.125 Millimolar.

Under the conditions above, RNA templates ligated to yield very accurate data while non-specific ligation background was significantly reduced. When the identical ligation mix was ligated at 42° C., efficiency of ligation dropped 10-100 fold to yield incorrect data.

It is evident from the above results and discussion that the subject invention provides improved highly sensitive methods of detecting nucleic acids, including siRNAs, in complex nucleic acid samples. Accordingly, the present invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of quantifying the amount of a target nucleic acid of less than about 30 nt in length in a sample, said method comprising:
a) contacting said sample with at least two ligation domains that are complementary to different domains of said target nucleic to produce a reaction mixture;
b) ligating any resultant annealed ligation domains of any resultant ligation oligonucleotide/target nucleic acid complexes in said reaction mixture to produce a pseudotarget nucleic acid; and
c) determining the presence of any pseudotarget nucleic acids in said reaction mixture to quantify the amount of said target nucleic acid in said sample.

2. The method according to claim 1, wherein said target nucleic acid is a ribonucleic acid.

3. The method according to claim 1, wherein said target nucleic acid does not exceed about 25 nt in length.

4. The method according to claim 1, wherein said target nucleic acid is single-stranded.

5. The method according to claim 1, wherein said target nucleic acid is an siRNA molecule.

6. The method according to claim 5, wherein said siRNA molecule is a shRNA molecule.

7. The method according to claim 1, wherein said ligation domains are present on separate oligonucleotides.

8. The method according to claim 7, wherein said ligation domains are present on a Combined Oligo.

9. The method according to claim 8, wherein said Combined Oligo is a linear deoxyribonucleic acid comprising terminal ligation domains.

10. The method according to claim 10, wherein said determining does not comprise amplifying said pseudotarget nucleic acid.

11. The method according to claim 1, wherein said determining comprises amplifying said pseudotarget nucleic acid.

12. The method according to claim 1, wherein said amplifying is by one of PCR, isothermal amplification, rolling circle amplification and branched DNA.

13. The method according to claim 1, wherein said quantifying is relative.

14. The method according to claim 1, wherein said quantifying is absolute.

15. The method according to claim 1, wherein said ligating occurs at a temperature ranging from about 20 to about 45° C.

16. The method according to claim 15, wherein said ligating occurs at a temperature ranging from about 37 to about 42° C.

17. The method according to claim 1, wherein said target nucleic acid is a peptide nucleic acid, locked nucleic acid, methylated nucleic acid, nucleic acid conjugate, thio-nucleic acid or morpholino nucleic acid.

18. A method of quantifying an siRNA in a sample, said method comprising:
a) contacting said sample with at least two ligation deoxyribo-oligonucleotides that are complementary to different adjacent domains of said siRNA to produce a reaction mixture;
b) ligating any annealed ligation deoxyribo-oligonucleotides of any resultant ligation deoxyribooligonucleotide/siRNA complexes in said reaction mixture to produce a pseudotarget nucleic acid;
c) amplifying any pseudotarget nucleic acids in said reaction mixture by PCR; and
d) detecting any resultant PCR amplified product to quantitate said siRNA in said sample.

19. The method according to claim 18, wherein said siRNA is single-stranded.

20. The method according to claim 18, wherein said siRNA is double-stranded.

21. The method according to claim 20, wherein said double-stranded siRNA is a short hairpin RNA.

22. The method according to claim 18, wherein said quantitating is relative.

23. The method according to claim 18, wherein said quantitating is absolute.

24. A system for detecting the presence of a target nucleic acid in a sample, said system comprising:
a) at least two ligation domains complementary to different regions of said target nucleic acid;
b) a ligase; and
c) pseudotarget detection reagents.

25. The system according to claim 24, wherein said pseudotarget detection reagents comprise PCR primers.

26. The system according to claim 25, wherein said system further comprises one or more additional PCR reagents.

27. A kit for detecting the presence of a target nucleic acid in a sample, said system comprising:
a) at least two ligation domains complementary to different regions of said target nucleic acid; and
b) instructions for using said at least two ligation oligonucleotides to practice a method according to claim 1.

28. The kit according to claim 27, said kit further comprising a ligase.

29. The kit according to claim 27, said kit further comprising pseudotarget detection reagents.

30. The kit according to claim 29, wherein said pseudotarget detection reagents comprise PCR primers.

31. The kit according to claim 30, wherein said system further comprises one or more additional PCR reagents.


SEQUENCE LISTING (9 sequences)

SEQ ID NO: 1 - RNA, 21 nt, Artificial Sequence
  Synthetic Construct
gcggagacagcgacgaagagc

SEQ ID NO: 2 - RNA, 21 nt, Artificial Sequence
  Synthetic Construct
gcucuucgucgcugucuccgc

SEQ ID NO: 3 - RNA, 50 nt, Artificial Sequence
  Synthetic Construct
gcggagacagcgacgaagagcuuccccucgcucuucgucgcugucuccgc

SEQ ID NO: 4 - DNA, 42 bp, Artificial Sequence
  Synthetic Construct
cggtattcggaatcttgccatggccggatccgcggagacagc

SEQ ID NO: 5 - DNA, 35 bp, Artificial Sequence
  Synthetic Construct
gacgaagagctttttttctcttaccagcctaactt

SEQ ID NO: 6 - DNA, 16 bp, Artificial Sequence
  Synthetic Construct
cggtattcggaatctt

SEQ ID NO: 7 - DNA, 16 bp, Artificial Sequence
  Synthetic Construct
aagttaggctggtaag

SEQ ID NO: 8 - DNA, 44 bp, Artificial Sequence
  Synthetic Construct
cggtattcggaatcttgccatgagcgagatgcagcggagacagc

SEQ ID NO: 9 - DNA, 29 bp, Artificial Sequence
  Synthetic Construct
gacgaagagctcttaccagcctaacttat